(19) United States
(12) Patent Application Publication
(10) Pub. No.: US 2005/0288238 A1
(43) Pub. Date: Dec. 29, 2005

(54) Benzocyclodecane derivatives with antitumor activity

(75) Inventors: Mauro Angiolini, Gavirate-VA (IT); Sylvie Ducki, Sannois (FR); Maria Menichincheri, Milano (IT); Nicola Mongelli, Milano (IT); Ermes Vanotti, Milano (IT); Marina Ciomei, Corsico (IT)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/878,212

(22) Filed: Jun. 28, 2004

(51) Int. Cl. A61K 31/704; C07H 15/24; A61K 31/225; C07C 67/02

(52) U.S. Cl. 514/33; 514/548; 536/18.1; 560/256

(57) ABSTRACT

A compound which is a benzocyclodecane of the formula I: 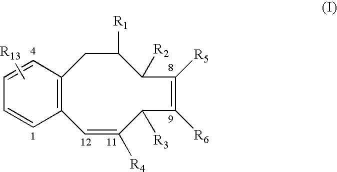
wherein: [symbol] at positions 8-9 and 11-12 independently represents a single or double bond,
—R₁ is ═O, or —OR₇, R₇ is H, C₁-C₇ alkanoyl, benzoyl, C₁-C₁₀ alkyl, C₂-C₁₀ alkenyl or COCH═CHR₈, R₈ is aryl or heterocyclyl; —R₂ and —R₃ are H, ═O or —OR₉, R₉ is H, C₁-C₇ alkanoyl or benzoyl; when at position 11-12 there is a single bond, then —R₄ represents
═O, ═CH₂, ═CHCOOR₁₀, R₁₀ is C₁-C₁₀ alkyl or aryl; ═CH(OCH₃), —OR₉; —CH₂OR₁₁, R₁₁ is H or a sugar residue, —COR₁₂, R₁₂ is H, —OH or —OR₁₀; or when at position 11-12 there is a double bond, then —R₄ is —CH₂OR₁₁ or —COR₁₂; —R₅ and —R₆ are H or, when at position 8-9 there is a single bond, taken together form a cyclopropane ring; R₁₃ is H or 1-3 substituents selected from C₁-C₆ alkyl, C₂-C₆ alkenyl, phenyl, phenyl C₁-C₆ alkyl, halogen, hydroxy, C₁-C₆ alkoxy, aryloxy, cyano, nitro, amino, C₁-C₁₀ alkylamino, arylamino, C₁-C₇ alkanoylamino, aroylamino, hydroxycarbonyl, aminocarbonyl, C₁-C₆ alkylcarbonyl,
   C₁-C₆ alkylaminosulfonyl and arylaminosulfonyl group; with the provisos that if R₁ and R₄ ═O, then one of R₂, R₃, R₅, R₆ and R₁₃ is not H atom; or a pharmaceutically acceptable salt thereof. These benzocyclodecane derivatives are endowed with antitumor activity; a process and new intermediates for their preparation, the pharmaceutical compositions containing them, and their use in the prevention, control and treatment of cancer are also provided.

### BACKGROUND OF INVENTION

The present invention relates to benzocyclodecane derivatives, to a process for their preparation, to pharmaceutical compositions containing them, and to the use of such compounds in the prevention, control and treatment of cancer.

In the field of antitumor compounds, a specific class comprises compounds from natural sources acting by mitotic arrest through induced tubulin polymerization. Examples of these natural products are paclitaxel, isolated from Taxus Brevifolia, Sarcodictyins A and B, isolated in 1987 by Pietra et al. from the Mediterranean stoloniferan coral Sarcodictyon roseum, and the diterpene glycoside eleutherobin, isolated from an Eleutherobia species of australian soft coral.

Now, there is a strong need for simplified molecules, which nevertheless maintain the useful properties referred to above characterizing the natural products.

In J. Chem. Soc. 1967 (7), 565-568 there is described the synthesis of benzocyclodecenone derivatives, whithout any suggestion on their pharmacoloigcal activity.

The present invention relates to a new class of antitumor compounds. In particular, the present invention provides a compound which is a benzocyclodecane of formula (I) 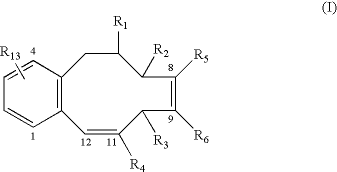
wherein:
[symbol] at positions 8-9 and 11-12 independently represents a single or double bond,
—R₁ represents oxygen (═O), or a residue —OR₇, wherein R₇ represents hydrogen, linear or branched C₁-C₇ alkanoyl, benzoyl, C₁-C₁₀ alkyl, C₂-C₁₀ alkenyl or a residue of the formula 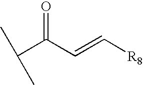
   wherein R₈ is an optionally substituted aryl or heterocyclyl;
—R₂ and —R₃ independently represents hydrogen, oxygen atom (═O) or a residue —OR₉, wherein R₉ represents hydrogen, C₁-C₇ alkanoyl or benzoyl;
oxygen atom (═O),
methylene (═CH₂),
═CHCOOR₁₀, wherein R₁₀ represents C₁-C₁₀ alkyl or optionally substituted aryl; ═CH(OCH₃),
or a residue of formula —OR₉, wherein R₉ is as defined above; —CH₂OR₁₁ wherein R₁₁ represents hydrogen or a sugar residue, —COR₁₂ wherein R₁₂ represents hydrogen, —OH or —OR₁₀, wherein R₁₀ is as defined above; or
when [symbol] at position 11-12 represents a double bond, then —R₄ represents a residue of formula —CH₂OR₁₁ or —COR₁₂ as defined above;
—R₅ and —R₆ are both hydrogen atoms or, when [symbol] at position 8-9 represents a single bond, taken together with the carbon atoms to which they are attached form a cyclopropane ring;
R₁₃ represents hydrogen or from one to three substituents selected from C₁-C₆ alkyl, C₂-C₆ alkenyl, optionally substituted phenyl, phenyl C₁-C₆ alkyl, halogen, hydroxy, C₁-C₆ alkoxy, aryloxy, cyano, nitro, amino, C₁-C₁₀ alkylamino, arylamino, C₁-C₇ alkanoylamino, aroylamino, hydroxycarbonyl, aminocarbonyl, C₁-C₆ alkylcarbonyl, C₁-C₆ alkylaminosulfonyl and arylaminosulfonyl group;
with the provisos that if R₁ and R₄ are both oxygen atom (═O), then one of R₂, R₃, R₅, R₆ and R₁₃ is not hydrogen atom; or a pharmaceutically acceptable salt thereof.

As used herein the terms “C₁-C₁₀ alkyl” and “C₁-C₆ alkyl” refer to a straight or branched chain alkyl moiety having respectively from 1 to 10 or from 1 to 6 carbon atoms, including for example, methyl, ethyl, propyl, isopropyl, n-butyl, i-butyl, sec-butyl, tert-butyl, n-pentyl, isopentyl, n-hexyl, n-heptyl and n-octyl.

The terms “C₂-C₁₀ alkenyl” and “C₂-C₆ alkenyl” as used herein refer to a straight or branched chain alkenyl moiety having respectively from 2 to 10 and from 2 to 6 carbon atoms and having in addition one double bond of either E or Z stereochemistry where applicable. Examples of alkenyl groups include: vinyl, allyl, metallyl, butenyl and crotyl. The term “aryl” as used herein refers to a monocyclic or bicyclic aromatic hydrocarbon group of 6 to 10 carbon atoms, such as phenyl, naphthyl, indanyl; furthermore, “aryl” as used herein may refer to a diphenyl group (—C₆H₄—C₆H₅). The term “C₁-C₇ alkanoyl” refers to acyl residues such as formyl, acetyl, and pentanoyl groups.

The term “heterocyclyl” as used herein refers to a 3- to 7-membered, substituted or unsubstituted, saturated or unsaturated heterocyclyl ring, containing at least one heteroatom selected from O, S and N, any ring carbon may be oxidized as a carbonyl, and wherein said heterocyclyl ring may be optionally fused to a second 5- or 6-membered, saturated or unsaturated heterocyclyl ring, or to a C₃-C₇ cycloalkyl ring, or to a benzene or naphthalene ring.

Examples of heterocyclyl groups are pyrrolyl, pyrrolidinyl, pyrazolyl, imidazolyl, triazolyl, tetrazolyl, oxazolyl, isoxazolyl, thiazolyl, isothiazolyl, thiadiazolyl, thienyl, tetrahydrothienyl, furyl, tetrahydrofuryl, aziridinyl, oxiranyl, azetidinyl, succinimido, pyridyl, piperidinyl, pyrazinyl, piperazinyl, pyridazinyl, hexahydropyridazinyl, pyrimidinyl, pyranyl, tetrahydropyranyl, benzothienyl, benzothiazolyl, benzoxazolyl, isobenzofuranyl, benzofuranyl, benzimidazolyl, indazolyl, chromenyl, indolyl, oxindolyl, phthalimido, 1-oxo-2-isoindolyl, quinolyl, isoquinolyl, tetrahydroisoquinolyl, indolizinyl, isoindolyl, 2-oxoisoindolyl, 1,2-(methylenedioxy)phenyl, quinuclidinyl, hydantoinyl, saccarinyl, cinnolinyl, purinyl, morpholinyl, thiomorpholinyl, dioxanyl, dithianyl and azepinyl.

Most preferred heterocyclyl groups are N-methyl-imidazolyl, 2-methyl-thiazolyl, 2-methyl-oxazolyl and pyridyl group. The term “C₃-C₇ cycloalkyl” as used herein refers to a 3- to 7-membered, substituted or unsubstituted, saturated or unsaturated carbon ring. Examples of cycloalkyl groups include: cyclopropyl, cyclobutyl, cyclopentyl and cyclohexyl groups. Preferably, when OR₁₁ is a sugar residue, it has the formula 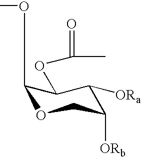
wherein Rₐ and R_{b} independently represent hydrogen, a hydroxy protecting group, or C₁-C₇ alkanoyl.

From all of the above, it is clear to the skilled man that any of the groups or substituents being defined, for instance, as alkoxy, alkylaminocarbonyl, alkylaminosulphonyl, arylaminosulphonyl and the like, have to be construed from the names of the groups from which they originate.

Substituents which may be present in the aryl or heterocyclyl groups in any of the above definitions of R₁-R₁₃ include the following:
halo (i.e., fluoro, bromo, chloro or iodo);
hydroxy;
nitro;
azido;
mercapto (i.e., —SH), and acetyl or phenylacetyl esters thereof (i.e., —SCOCH₃ and —SCOCH₂C₆H₅);
amino (i.e., —NH₂ or —NHR^{I} or —NR^{I}R^{II}, wherein R^{I} and R^{II}, which are the same or different, are straight or branched C₁-C₆ alkyl, phenyl, biphenyl (i.e., —C₆H₄—C₆H₅), or benzyl groups, optionally substituted by hydroxy, methoxy, methyl, amino, methylamino, dimethylamino, chloro or fluoro; or R^{I} and R^{II} taken together with the nitrogen atom to which they are attached form a heterocyclic ring such as morpholino, pyrrolidino, piperidino, pyperazino or N-methylpyperazino;
guanidino, i.e., —NHC(═NH)NH₂;
formyl (i.e.—CHO);
cyano;
carboxy (i.e.—COOH), or esters thereof (i.e., —COOR^{I}), or amides thereof (i.e., —CONH₂, —CONHR^{I} or —CONHR^{I}R^{II}), wherein R^{I} and R^{II} are as defined above, and including morpholino-amides, pyrrolidino-amides, and carboxymethylamides —CONHCH₂COOH;
sulfo (i.e., —SO₃H);
acyl, i.e., —C(O)R^{I}, wherein R^{I} is as defined above, including monofluoroacetyl, difluoroacetyl, trifluoroacetyl;
carbamoyloxy (i.e., —OCONH₂) and N-methylcarbamoyloxy;
acyloxy, i.e., —OC(O)R^{I} wherein R^{I} is as defined above, or formyloxy;
acylamino, i.e., —NHC(O)R^{I}, or —NHC(O)OR^{I}, wherein R^{I} is as defined above or is a group —(CH₂)ₜCOOH where t is 1, 2 or 3;
ureido, i.e., —NH(CO)NH₂, —NH(CO)NHR^{I}, —NH(CO)NR^{I}R^{II}, wherein R^{I} and R^{II} are as defined above, including —NH(CO)-(4-morpholino), —NH(CO)-(1-pyrrolidino), —NH(CO)-(1-piperazino), —NH(CO)-(4-methyl-1-piperazino);
sulfonamido, i.e., —NHSO₂R^{I} wherein R^{I} is as defined above;
a group —(CH₂)ₜCOOH, and esters and amides thereof, i.e., —(CH₂)ₜCOOR and —(CH₂)ₜCONH₂, —(CH₂ₜCONHR^{I}, —(CH₂)ₜCONR^{I}R^{II}, wherein t, R^{I} and R^{II} are as defined above;
a group —NH(SO₂)NH₂, —NH(SO₂)NHR^{I}, —NH(SO₂)NR^{I}R^{II}, wherein R^{I} and R^{II} are as defined above, including —NH(SO₂)-(4-morpholino), —NH(SO₂)-(1-pyrrolidino), —NH(SO₂)-(1-piperazino), —NH(SO₂)-(4-methyl-1-piperazino);
a group —OC(O)OR^{I}, wherein R^{I} is as defined above;
a group —OR^{I}, wherein R^{I} is as defined above, including —OCH₂COOH;
a group —SR^{I}, wherein R^{I} is as defined above, including —SCH₂COOH;
a group —S(O)R^{I}, wherein R^{I} is as defined above;
a group —S(O₂)R^{I}, wherein R^{I} is as defined above;
a group —SO₂NH₂, —SO₂NHR^{I}, or —SO₂NR^{I}R^{II}, wherein R^{I} and R^{II} are as defined above;
C₁-C₆ alkyl or C₂-C₆ alkenyl;
C₃-C₇ cycloalkyl;
substituted methyl selected from chloromethyl, fluoromethyl, difluoromethyl, trifluoromethyl, aminomethyl, N,N-dimethylaminomethyl, azidomethyl, cyanomethyl, carboxymethyl, sulfomethyl, carbamoylmethyl, carbamoyloxymethyl, hydroxymethyl, methoxycarbonylmethyl, ethoxycarbonylmethyl, tert-butoxycarbonylmethyl and guanidinomethyl.

When present, carboxy, hydroxy, mercapto and amino groups may be either free or in a protected form. Protected forms of said groups are any of those generally known in the art. Preferably, carboxy groups are protected as esters thereof, in particular methyl, ethyl, tert-butyl, benzyl, and 4-nitrobenzyl esters. Preferably, hydroxy groups are protected as silyl-ethers, ethers or esters thereof, in particular trimethyl silyl, tert-butyidiphenyl silyl, triethyl silyl, triisopropyl silyl or tert-butyidimethylsilyl ethers, methoxymethyl ethers, tetrahydropyranyl ethers, benzyl ethers, acetates or benzoates. Preferably, mercapto groups are protected as thioethers or thioesters, in particular tert-butyl thioethers, thioacetates or thiobenzoates. Preferably, amino groups are protected as carbamates, e.g. tert-butoxycarbonyl derivatives, or as amides, e.g. acetamides and benzamides.

As stated above, the present invention provides the salts of those compounds of formula (I) that have salt-forming groups, especially the salts of the compounds having a carboxylic group or the salts of the compounds having a basic group, especially an amino. The salts are especially physiologically tolerable salts, for example alkali metal and alkaline earth metal salts (e.g. sodium, potassium, lithium, calcium and magnesium salts), ammonium salts and salts with an appropriate organic amine or amino acid (e.g. arginine, procaine salts), and the addition salts formed with suitable inorganic acids (e.g. hydrochlorides, hydrobromides, sulfates, phosphates) or carboxylic and sulfonic organic acids (e.g. acetates, trifluoroacetates, citrates, succinates, malonates, lactates, tartrates, fumarates, maleates, methanesulfonates, p-toluenesulfonates).

Furthermore, hydrates, solvates of compounds of formula (I), and physiologically hydrolysable derivatives (i.e., prodrugs) of compounds of formula (I) are included within the scope of the present invention.

It is to be noted that the R₁, R₂, R₃, R₄, R₅ and R₆ substituents may be above or under the plane, so that the present invention encompasses all the possible stereo isomers (e.g. diastereoisomers, epimers, geometrical isomers) of the compounds of formula (I), as well as their racemic or optically active mixtures related to these substituents.

In the preferred configuration R₁, which is the substituent at ring position 6, is under the plane:

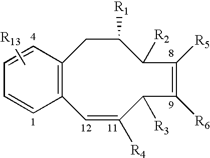

In a preferred compound of the present invention, the benzocyclodecane has the following formula (IA): 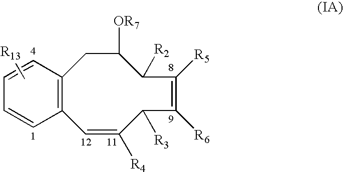
wherein:
[symbol] at positions 8-9 and 11-12 independently represents a single or double bond, R₇ represents a residue of the formula 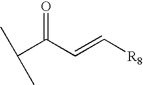
   wherein R₈ is N-methyl imidazolyl, phenyl, methyl-thiazolyl, methyl-oxazolyl or pyridyl group;
one of —R₂ and —R₃ represents hydrogen and the other one is hydrogen or oxygen (═O), hydroxy or acetoxy group;
when [symbol] at position 11-12 represents a single bond, then —R₄ represents oxygen (═O), methylene (═CH₂), ═CHCOOR₁₀, wherein R₁₀ represents methyl or ethyl, ═CH(OCH₃), —CHO, hydroxy, acetoxy, or —CH₂OR₁, wherein R₁₁ represents hydrogen or a sugar residue having the formula 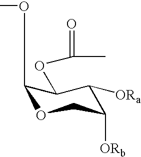
   wherein Rₐ and R_{b} independently represent hydrogen, a hydroxy protecting group, or C₁-C₇ alkanoyl, or

when [symbol] at position 11-12 represents a double bond, then —R₄ represents a residue of formula —CO₂C₂H₅; and
—R₅ and —R₆ are both hydrogen atoms or, when [symbol] at position 8-9 represents a single bond, taken together with the carbon atoms to which they are attached form a cyclopropane ring;
R₁₃ represents hydrogen atom, two methyl groups at positions 1 and 4, one methyl group at position 4 and one isopropyl group at position 1.

The present invention also provides a process for preparing a compound of the invention as defined above, which process comprises:
cyclizing a compound of formula II 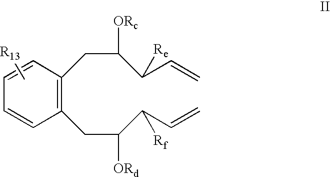
   wherein R_{c} represents hydrogen, a hydroxy protecting group, C₁-C₇ alkanoyl or benzoyl or, taken together with Rₑ, forms an acetonide ring; R_{d} represents hydrogen, a hydroxy protecting group, C₁-C₆ alkanoyl, or benzoyl, or, taken together with R_{f}, forms an acetonide ring; Rₑ represents hydrogen atom and R_{f} represents hydrogen atom or a free or protected hydroxy group, or is linked to the adjacent OR_{d} substituent as defined above; R_{f} represents hydrogen atom and Rₑ represents hydrogen atom or a free or protected hydroxy group or is linked to the adjacent OR_{c} substituent as defined above;
and, if desired, converting the resultant compound of formula I′, 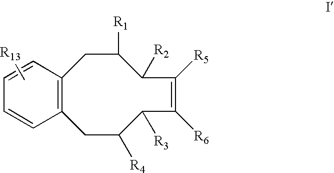
   wherein R₁ is OR_{c}, R₂ is Rₑ, R₃ is R_{f}, R₄ is OR_{d}, in which R_{c}, R_{d}, Rₑ and R_{f} are as defined above and R₅ and R₆ are hydrogen atoms, into another different compound of formula I as defined above; and/or if desired, converting a compound of formula I′ or I into a pharmaceutically acceptable salt therof; and/or, if desired converting a pharmaceutically acceptable salt of a compound of formula I or I′ into the corresponding free compound.

Preferably, the hydroxy protecting groups are silyl or methoxymethyl group; R_{c} represents a C₁-C₆ alkanoyl group, more preferably an acetyl group, or a silyl protecting group, more preferably a t-butyldiphenylsilyl group. The cyclization to give the compound of formula I′ as single Z isomer can be performed through the Ring Closing Metathesis (RCM) reaction. In particular, the RCM reaction is carried out in the presence of an appropriate catalyst, more preferably a Nolan and Grubb's catalyst, described for example in J. Am. Chem. Soc., 1999, 121, 2674 and in Org. Lett., 1999, 1, 953.

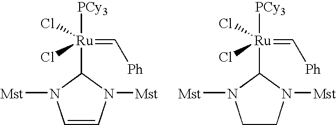

RCM Catalyst A RCM Catalyst B

[Mst=C₆H₂-2,4,6-(CH₃)₃]

The conversion of a compound of formula I′ or I into another different final compound of formula I may be carried out in several ways, depending on the meanings of the substituents and the presence of the unsaturated bonds in the ring. Such conversions follow conventional procedures known in the art.

For example, a compound of formula I wherein —R₁ represents a residue of the formula 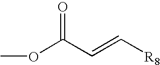
wherein R₈ is as defined above, can be obtained by condensing a corresponding compound of the formula I or I′ wherein —R₁ represents hydroxy group with a the appropriate derivative of formula III 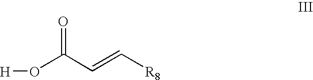
wherein R₈ is as above defined. These compounds of formula III are known or can be prepared according to known procedures.

Therefore, it is a further object of the present invention a process for obtaining a compound of formula I″′ 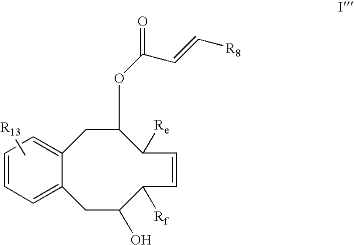
wherein Rₑ, R_{f}, R₁₃ and R₈ are as defined above, which process comprises deprotecting a compound of formula I″: 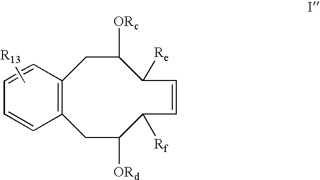
wherein R_{c}, Rₑ, R_{d}, R_{f} and R₁₃ are as defined above, condensing the resultant compound of formula Iᵢᵥ 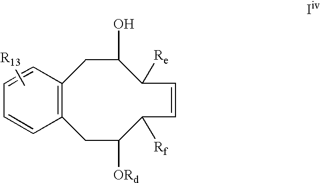
wherein Rₑ, R_{d}, R_{f} and R₁₃ are as defined above, with a compound of formula III or an activated form thereof: 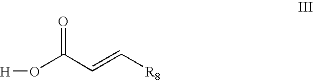
wherein R₈ is as above defined, optionally in presence of a condensing agent; and, if necessary, deprotecting the resultant compound of formula I^{v}. 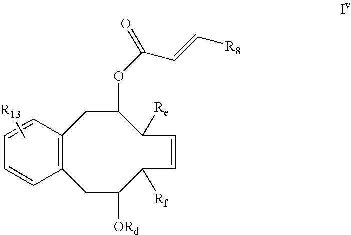
wherein Rₑ, R_{d}, R_{f}, R₈ and R₁₃ are as defined above, and R_{d} represents a hydroxy protecting group, C₁-C₆ alkanoyl, or benzoyl, or, taken together with R_{f}, forms an acetonide ring; to give the desired compound of formula I″′ as above defined.

As a more specific example, the process for preparing a compound of formula I wherein —R₁ represents a residue of the formula 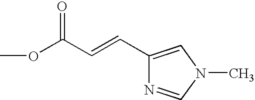
is depicted in the scheme 1 below:

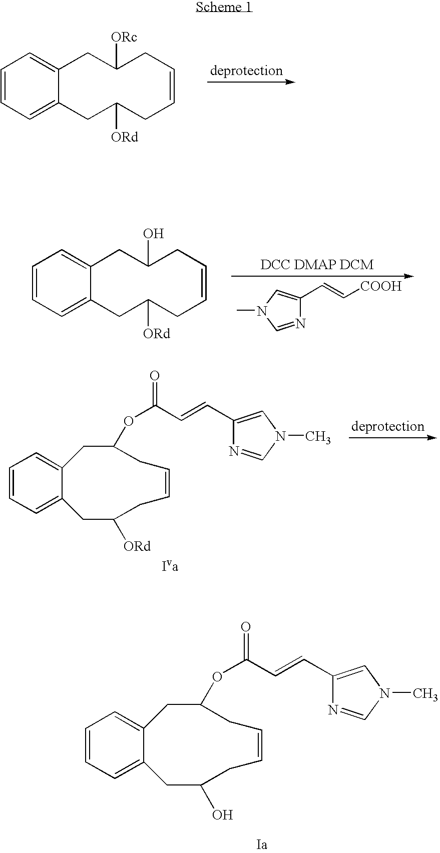

The reaction with (E)-N-methylurocanic acid can be carried out in dichloromethane (DCM) in presence of dicyclohexylcarbodiimmide (DCC) and 4-dimethylaminopyridine (DMAP). The deprotection steps can be basic hydrolysis in case Rc and/or Rd are acetyl groups.

A compound of formula I wherein —R₂, —R₃ or —R₄ represents an oxygen atom ═O can be obtained from a corresponding compound of formula I or I′ as defined above wherein —R₂, —R₃ or —R₄ represents a hydroxy group by means of oxidation, for example with Dess-Martin periodinane, pyridinium dichromate (PDC) or pyridinium chlorochromate (PCC) or under Swern oxidation conditions (dimethylsulfoxide/oxalyl chloride), provided that the other hydroxy groups in the molecule, if any, are protected. A compound of formula I wherein —R₄ represents an oxygen atom ═O can be conveniently converted into a corresponding compound of formula I wherein —R₄ represents methylene (═CH₂), ═CHCOOR₁₀ wherein R₁₀ is as defined above, or ═CH(OCH₃) by reaction with a suitable Wittig reagent, such as for example, respectively, Ph₃P═CH₂, Ph₃P═CHCOOR₁₀, wherein R₁₀ is as defined above and Ph₃P═CH(OCH₃). A compound of formula I wherein —R₄ represents ═CH(OCH₃) can be then converted by acidic hydrolysis into a corresponding compound of formula I wherein —R₄ represents —CHO, which in turn may be either reacted with a reducing agent to give a compound of formula I wherein —R₄ represents —CH₂OH, or oxidised with a suitable reagent such as NaClO₂ to give a compound of formula I wherein —R₄ represents —COOH. A compound of formula I wherein —R₄ represents an oxygen atom ═O can also be converted into a compound of formula I wherein —R₄ represents a —COOR₁₀ group wherein R₁₀ is as defined above and the bond at position 11-12 is double by treatment with triflic anhydride in the presence of a base followed by reaction of the resultant enol-triflate with CO and R₁₀—OH wherein R₁₀ is as defined above in the presence of Palladium catalyst and a base such as triethylamine according to known procedures as those described in J. Chem. Soc. Perkin Trans. I, 1991 (5), 969-979. Such compounds of formula I wherein —R₄ represents a —COOH group and the bond at position 11-12 is double can be converted by selective reduction into the corresponding 11-12 unsaturated compounds of formula I wherein —R₄ represents a —CH₂OH group, for example by treatment with ClCOOEt/NaBH₄.

A compound of formula I′ or I wherein the bond at position 8-9 is double may be converted into the corresponding compound of formula I with a single bond at the 8-9 position and wherein R₅ and R₆ are hydrogen atoms by hydrogenation, such as by treatment with H₂ and a suitable catalyst like a Palladium on charcoal catalyst according to the methods known in the art; or into the corresponding compounds of formula I with a single bond at the 8-9 position wherein R₅ and R₆ taken together with the carbon atoms to which they are attached, form a cyclopropane ring by treatment with a suitable reactant such as a zinc carbenoid (J. Am. Chem. Soc. 2001, 123, 8139-8140).

A compound of formula I may be converted into a pharmaceutically acceptable salt thereof using conventional techniques. Suitable salts include those mentioned above.

A compound of the formula II may be prepared as described in any one of the following schemes, in which R_{c}, R_{d}, Rₑ, R_{f} and R₁₃ have the meanings above defined:

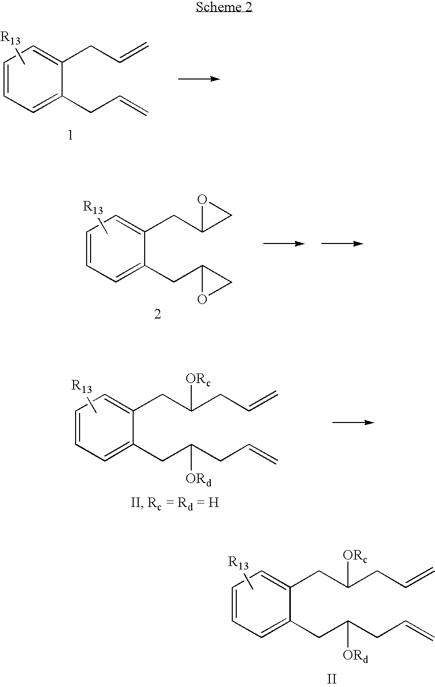

Compound 1 where R₁₃ represents hydrogen atom is known and can be prepared according to known procedures (Tetrahedron Lett. (2000), 41(5), 729-731). Compound 1 can also be obtained by the copper mediated reaction of a vinyl organometallic reagent, such as vinyl magnesium bromide, with the appropriate 1,2 dibromomethyl-phenyl derivative (see for example J. Agric. Food Chem. 45, 1422, 1997). Compound 1 can be conveniently transformed into compound 2 by oxidation, for example by treatment with an inorganic or organic peracid, such as meta-chloroperbenzoic acid, and then compound 2 can be converted into the compound II, wherein R_{c} and R_{d} are both hydrogen atoms, by the addition of a vinyl organometallic reagent, such as vinyl magnesium bromide. The resultant compound II is then protected to yield the desired compound of formula II wherein R_{c} and R_{d} are hydroxy protecting groups as defined above. By the above process, for example, there are obtained compounds of formula II wherein R_{c} and R_{d} are both acetyl groups and R_{d} and R_{f} are hydrogen atoms. It is a further object of the present invention an intermediate compound of formula II 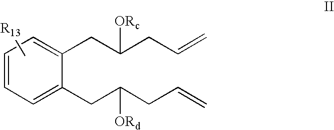
wherein R_{c} and R_{d} are hydrogen atoms or hydroxy protecting groups, and R₁₃ has the meanings above defined.

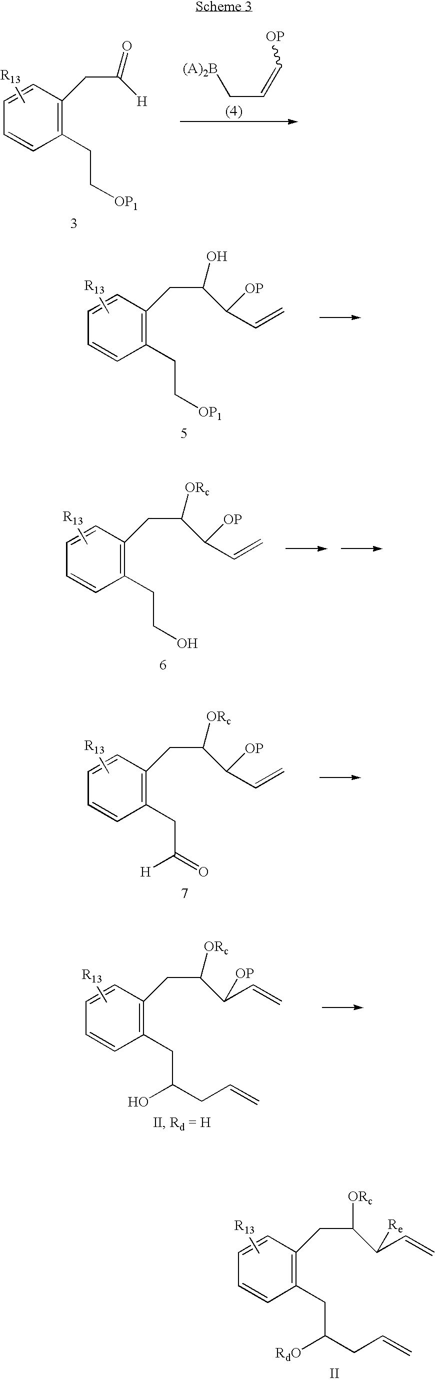

Compound 3 wherein R₁₃ represents hydrogen atom and P₁ represents acetyl group is known, other compounds 3 can be analogously prepared as described in the literature (Tetrahedron 1988, 44, 7027). To the properly protected compound 3, wherein P₁ represents a hydroxy protecting group such as an acetyl or a silyl protecting group, is added the appropriate allylic boronate of formula (4) wherein P represents a hydroxy protecting group and A represents a suitable organic residue. These compounds of formula (4) are known or can be prepared according to known procedures. Depending on the Z or E stereochemistry of the starting allylic boronate (4) in scheme 3, both syn and anti allylic derivatives 6 can be obtained. Alternatively, a Compound 3 can be submitted to Brown's stereoselective allylation reaction, (J. Org. Chem. 1982, 47, 5065). In this case the desired stereochemistry of the two oxygenated vicinal substituents can be controlled in the resultant compound of formula 5 just by choosing the suitable absolute stereochemistry of an alpha-pinene-derived allylic reagent (4), wherein A represents I-Ipc from (−)-alpha-pinene or d-Ipc from (+)-alpha-pinene.

All possible stereoisomers can be synthesized as a mixture and obtained as single stereoisomers also by chromatographic separation. In particular enantiomers can be obtained by chiral chromatographic separation (by using for example chiral solid support).

Compound 5 is protected (introduction of R_{c} group) and then deprotected (removal of P₁) to yield Compound 6, that is then oxidized to give the aldehyde derivative 7, for example under Swern oxidation conditions (dimethylsulfoxide/oxalyl chloride) or with PCC. Addition to the Compound 7 of an allylic organometallic species (for example allyl magnesium bromide) affords the compound II (R_{d}═H), that is suitably protected to be converted into another compound II. By the above process, for example, there are obtained compounds of formula II wherein R_{c}, R_{d} and Rₑ are hydroxy protecting groups and R_{f} is hydrogen atom.

### Biological Tests

Microtubule assembly and disassembly assay.

Pig brain tubulin was prepared by two cycles of assembly and disassembly and it was stored in liquid nitrogen in Microtubule Assembly Buffer (MAB: 0.1 M MES, 2.5 mM EGTA, 0.5 mM MgSO₄, 0.1 mM EDTA, 0.1 mM DTT pH 6.4). Assembly was monitored by the method of Gaskin et al. (Gaskin F, Cantor C R, Shelanski M L, 1974,: Turbidimetric studies of the in vitro assembly and disassembly of porcine neurotubules. J. Mol. Biol. 89: 737-758). The cuvette (1 cm path) containing 0.5 mg/ml tubulin and 1 mM GTP was shifted to 37° C. and continuous turbidity measurements were made at 340 nm on a spectrophotometer equipped with an automatic recorder and a thermostatically regulated sample chamber. After 30 min CaCl₂ (5 mM) was added and disassembly was monitored for 10 min as decreased turbidity. Scalar doses of test compounds were monitored at regular intervals of 15 min.

Data were expressed as percentage of reassembly induced by the tested compounds and the dose effecting tubulin assembly by 90% at 37° C. (ED₉₀) was calculated on this curve.

The compound I^{v} a prepared in Example 9 showed an ED₉₀ of 10 microM.

### Cytotoxicity

A2780 cells (2000/well) were seeded in multi-well plates (96 wells) in the presence of 200 μl of the complete medium RPMI 1640+10% FCS. After 24 h, the cells were treated with the compounds: the compounds' solution (200×) was prepared in DMSO 100% and 1 μl/well was added. 5 scalar concentrations for each compound were tested in four replicates. The cells were incubated at 37° C., 5% CO₂ for 72 h.

Colorimetric assay (SRB: sulforhodamine B): cell cultures were fixed with trichloroacetic acid, stained with 0.4% SRB dissolved in 1% acetic acid. Unbound dye was removed by four washes with 1% acetic acid and protein-bound dye was extracted with 10 mM Tris base for determination of optical density in a 96-well microtiter plate reader. IC₅₀ and IC₉₀ (concentration inhibiting cell proliferation by 50 or 90%) were determined by data analysis in the Microsoft Excel 97 program.

### Effect on Cell Cycle Progression

Human colon carcinoma HCT116 cells were seeded in culture flasks and treated 24 h after incubation at 37° C. At the end of the treatment (24 or 48 or 72 hours), cells were counted and resuspended in propidium iodide (PI) staining solution (0.1% sodium citrate, 0.1% nonidet P40, 6.5 μg/ml Rnasi A, 50 μg/ml PI). After incubation in the dark at room temperature for at least 30 minutes, samples were then analyzed for cell cycle on FacScan (Becton Dickinson) flow cytometer.

Compounds of formula I of the invention show enhanced antitumor activity and acceptable toxicity. They are useful as antitumour agents in the prevention, treatment and/or control of cancer, for instance in the treatment of leukemia and solid tumors, such as colon, colo-rectal, ovarian, mammary, prostate, lung, kidney and also melanoma tumors. A human can be treated by a method comprising administering thereto a therapeutically effective amount of a compound of the invention. The invention therefore provides a method of treating a patient in need of an antitumour agent, which method comprises the administration thereto of a compound as defined above. The condition of the human patient can thus be improved. The invention also provides the use of a compound of the invention as defined above in the manufacture of a medicament for use as an antitumour agent.

The dosage range adopted will depend on the route of administration and on the age, weight and condition of the patient being treated. The compound of formula (I) is typically administered by parenteral route, for example intramuscularly, intravenously or by bolus infusion. A suitable dose range is from 1 to 1000 mg of equivalent per m² body surface area of active drug, for instance from 10 to 500 mg/m².

The compounds of formula (I) may be formulated into a pharmaceutical composition together with a pharmaceutically carrier or diluent. The invention therefore further provides a pharmaceutical composition which comprises a pharmaceutically acceptable diluent or carrier and, as an active ingredient, a compound as defined above. The pharmaceutical compositions of the invention are prepared by conventional methods and are administered in a pharmaceutically acceptable form. For example, the solid oral forms may contain, together with the active compound, diluents, e.g. lactose, dextrose, saccharose, sucrose, cellulose, corn starch or potato starch; lubricants, e.g. silica, talc, stearic, magnesium or calcium stearate, and/or polyethylene glycols; binding agents, e.g. starches, arabic gum, gelatine, methylcellulose, carboxymethylcellulose or polyvinyl pyrrolidone; disaggregating agents, e.g. a starch, alginic, alginates or sodium starch glycolate; effervescing mixtures; dyestuffs; sweeteners; wetting agents such as lecithin, polysorbates, laurylsulphates; and, in general, non-toxic and pharmacologically inactive substances used in pharmaceutical formulations. Said pharmaceutical preparations may be manufactured in known manner, for example, by means of mixing, granulating, tabletting, sugar-coating, or film-coating processes.

The liquid dispersions for oral administration may be e.g. syrups, emulsions and suspensions.

The syrups may contain as carrier, for example, saccharose or saccharose with glycerine and/or mannitol and/or sorbitol.

The suspensions and the emulsions may contain as carrier, for example, a natural gum, agar, sodium alginate, pectin, methylcellulose, carboxymethylcellulose, or polyvinyl alcohol.

The suspension or solutions for intramuscular injections may contain, together with the active compound, a pharmaceutically acceptable carrier, e.g. sterile water, olive oil, ethyl oleate, glycols, e.g. propylene glycol, and, if desired, a suitable amount of lidocaine hydrochloride. The solutions for intravenous injections or infusions may contain as carrier, for example, sterile water or preferably they may be in the form of sterile, aqueous, isotonic saline solutions or they may contain as a carrier propylene glycol.

The suppositories may contain together with the active compound a pharmaceutically acceptable carrier, e.g. cocoa butter, polyethylene glycol, a polyoxyethylene sorbitan fatty ester surfactant or lecithin.

Typically the pharmaceutical compositions are formulated for parenteral administration, for example by dissolution in water for injection or physiological saline.

The following examples illustrate the invention without limiting it.

### EXAMPLE 1

### 1,2-Diallyl-benzene

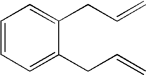

A 1.0 M tetrahydrofurane (THF) solution of vinyl bromide (300 ml, 0.30 mol) was added to a flask containing magnesium turnings (7.01 g, 0.29 mol) in freshly dried THF (100 ml) under an atmosphere of nitrogen. The mixture was heated under reflux until all the magnesium disappeared (2 hours) and copper (I) iodide (28.5 g, 0.15 mol) was added drop-wise to the resulting slurry keeping the temperature below −30° C. A solution of 6′-dibromoxylene (13.75 g, 0.052 mol) in dry THF (100 ml) was then slowly dropped into the green slurry at −60° C. The resulting mixture was stirred at −60° C. for 1 hour and at 0° C. for a further 3 hours. When TLC analysis showed no starting material left, the mixture was quenched with a saturated solution of ammonium chloride (100 ml) and extracted with diethyl ether (3×100 ml). The etheral layer was dried over Na₂SO₄, filtered and evaporated (no heating, the product is volatile). Flash chromatography (silicagel, hexane) afforded the title compound 1,2-diallyl-benzene in 77% yield (6.3 g); δ_{H} (300 MHz, CDCl₃) 7.18 (4H, m, Ph), 5.90-6.05 (2H, m, 2×CH═), 4.97-5.10 (4H, m, 2×CH₂═), 2.40 (4H, m, 2×CH₂).

### EXAMPLE 2

### 2-[2-(oxiran-2-ylmethyl)benzyl]oxirane

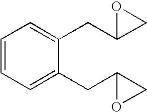

To a solution of 1,2-diallyl-benzene prepared in Example 1 (0.55 g, 3.48 mmol) in dry DCM (50 ml) was added m-chloroperoxybenzoic acid (3.15 g, 9.10 mmol). The mixture was stirred at RT under an atmosphere of nitrogen for 16 hours. A saturated solution of NaHCO₃ (50 ml) was added and the mixture was stirred for a further 15 minutes. The organic layer was then dried over Na₂SO₄, filtered and evaporated. The residue was purified by flash chromatography (silicagel, 10% ethyl acetate in hexane) to give 2-[2-(oxiran-2-ylmethyl)benzyl]oxirane in 82% yield (0.54 g); δ_{H} (300 MHz, CDCl₃) 7.20-7.30 (4H, m, Ph), 3.12-3.22 (2H, m, 2×CH), 2.94 (4H, m, 2×CH₂), 2.80 (2H, m, 2×CHaHb), 2.53 (2H, m, 2×CHaHb); m/z 208.3 (M+NH₄⁺, 100%), 191.3 (M+H⁺, 10%).

### EXAMPLE 3

### 1-[2′-(2″-Hydroxy-Pent-4″-enyl)-phenyl]-pent-4-en-2-ol

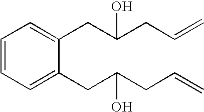

A 1.0 M THF solution of vinyl bromide (220 ml, 0.22 mol) was added to magnesium turnings (5.0 g, 0.21 mol) in freshly dried THF (80 ml) under an atmosphere of nitrogen. The mixture was heated under reflux until all the magnesium disappeared. Copper (I) iodide (19.58 g, 0.10 mol) in dry THF (50 ml) was added drop-wise to the vinyl magnesium bromide at −50° C. and the greenish slurry was stirred for 10 minutes. 2-[2-(oxiran-2-ylmethyl)benzyl]oxirane prepared in Example 2 (3.9 g, 0.02 mol) in dry THF (50 ml) was added drop-wise to the slurry keeping the temperature below −65° C., stirred 1 hour at this temperature and at 0° C. until all starting material disappeared by TLC analysis. The mixture was then quenched with a saturated solution of ammonium chloride and extracted with diethyl ether (3×75 ml). The etheral layer was filtered through a 5-cm pad of silicagel, dried over Na₂SO₄, filtered and evaporated to dryness. The residue was purified by flash chromatography (silicagel, 10% ethyl acetate in hexane) to furnish 1-[2′-(2″-hydroxy-pent-4″-enyl)-phenyl]-pent-4-en-2-ol in 85% yield (4.3 g) as a yellowish powder; δ_{H} (300 MHz, CDCl₃) 7.21 (4H, m, Ph), 5.90 (2H, m, 2×CH═), 5.18 (4H, m, 2×CH₂), 3.89 (2H, q, J 6 Hz, CH), 2.83 (4H, d, J 6 Hz, CH₂), 2.37 (4H, m, 2×CH₂), 2.23 (2H, bs, 2×OH); m/z 305.3 (M+CH₃COO⁻, 100%), 264.3 (M+NH₄⁺, 100%), 247.3 (M+H+, 60%).

### EXAMPLE 4

### Acetic acid 1-[2′-(2″-acetoxy-pent-4″-enyl)-benzyl]-but-3-enyl ester

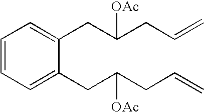

A solution of 1-[2′-(2″-hydroxy-pent-4″-enyl)-phenyl]-pent-4-en-2-ol prepared in Example 3 (0.57 g, 2.32 mmol), acetic anhydride (1 ml), pyridine (0.5 ml), 4-dimethylaminopyridine (2 mg) in dichloromethane (DCM, 20 ml) was stirred at room temperature (RT) for 4 hours, washed with a saturated solution of NaHCO₃ (20 ml), water (20 ml), dried over Na₂SO₄, filtered and concentrated. The residue was purified by flash chromatography (silicagel, 10% ethyl acetate in hexane) to furnish the desired acetic acid 1-[2′-(2″-acetoxy-pent-4″-enyl)-benzyl]-but-3-enyl ester in 89% yield (0.68 g); δ_{H} (300 MHz, CDCl₃) 7.13 (4H, s, Ph), 5.78 (2H, m, 2×CH═), 5.10 (4H, m, 2×CH₂═), 3.73 (2H, m, 2×CH), 2.92 (4H, d, J 7 Hz, 2×CH₂), 2.35 (4H, m, 2×CH₂).

### EXAMPLE 5

### Acetic acid 11-acetoxy-5,6,7,10,11,12-hexahydro-benzocyclodec-8-en-6-yl ester

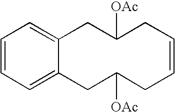

To a solution of acetic acid 1-[2′-(2″-acetoxy-pent-4″-enyl)-benzyl]-but-3-enyl ester prepared in Example 4 (0.68 g, 2.06 mmol) in dry DCM (200 ml) was added Grubbs II catalyst B (35.4 mg, 2 mol %). The flask was flushed with nitrogen and the pink solution was stirred at RT under an atmosphere of nitrogen for 2 hours. After stirring at ambient air until the solution turned brown (decomposed catalyst), the solvent was evaporated. The residue was purified by flash chromatography (silicagel, 10% ethyl acette in hexane) to furnished the cis-cyclized product acetic acid 11-acetoxy-5,6,7,10,11,12-hexahydro-benzocyclodec-8-en-6-yl ester in 74% yield (0.46 g); δ_{H} (300 MHz, CDCl₃) 7.29 (2H, m, Ph), 7.19 (2H, m, Ph), 5.78 (2H, m, 2×CH═), 5.30 (2H, m, CH), 3.04 (2H, t, J 12 Hz, 2×CHaHb), 2.75 (2H, dd, J 5, 12 Hz, 2×CHaHb), 2.04-2.18 (10H, m, 2×CH₂, 2×CH₃); m/z (EI) 302 (M⁺, 10%), 242 [(M−CH₃COOH)⁺, 25], 182 [(M−2×CH₃COOH)⁺, 55], 43 (CH₃CO⁺, 100); X-ray.

### EXAMPLE 6

### Acetic acid 11-hydroxy-5,6,7,10,11,12-hexahydro-benzocyclodec-8-en-6-yl ester

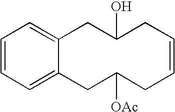

To a stirred solution of acetic acid 11-acetoxy-5,6,7,10,11,12-hexahydro-benzocyclodec-8-en-6-yl ester prepared in Example 5 (30.6 mg, 0.101 mmol) in dry methanol (5 ml) was added potassium carbonate (13.2 mg, 0.096 mmol). After 30 minutes, the solution was quenched with water (10 ml), acidified with 1N HCl and extracted with DCM (2×10 ml). The organic layer was dried over Na₂SO₄, filtered and evaporated. Flash chromatography (silicagel, 10% ethyl acetate in hexane) afforded acetic acid 11-hydroxy-5,6,7,10,11,12-hexahydro-benzocyclodec-8-en-6-yl ester as a white powder in 84% yield (22 mg); δ_{H} (300 MHz, CDCl₃) 7.10-7.30 (4H, m, Ph), 5.77-5.99 (2H, m, 2×CH═), 5.23 (1H, m, CH), 4.22 (1H, m, CH), 2.95-3.17 (2H, m, CH₂), 2.88 (2H, m, CH₂), 2.19 (3H, s, CH₃), 2.02-2.18 (4H, m, 2×CH₂); m/z 319.3 [(M+CH₃COO⁻, 100%), 278.3 [(M+NH₄)⁺, 100%].

### EXAMPLE 7

### Acetic acid 11-oxo-5,6,7,10,11,12-hexahydro-benzocyclodecen-6-yl ester

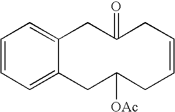

To a solution of acetic acid 11-acetoxy-5,6,7,10,11,12-hexahydro-benzocyclodec-8-en-6-yl ester prepared in Example 5 (110.2 mg, 0.365 mmol) in dry methanol (10 ml) was added potassium carbonate (49.7 mg, 0.360 mmol). The solution was stirred at RT for 30 minutes, quenched with water (10 ml) and extracted with DCM (2×20 ml). The organic layer was dried over Na₂SO₄, filtered and evaporated to give crude acetic acid 11-hydroxy-5,6,7,10,11,12-hexahydro-benzocyclodec-8-en-6-yl ester. The crude acetic acid 11-hydroxy-5,6,7,10,11,12-hexahydro-benzocyclodec-8-en-6-yl ester was redissolved in DCM (10 ml) and pyridinium chlorochromate (78.2 mg, 0.363 mmol) was added. The mixture was stirred at RT for 2 hours, filtered and evaporated. The residue was purified by flash chromatography (silicagel, 5% ethyl acetate in hexane) to afford the tiltle compound in 46% yield (43 mg); δ_{H} (300 MHz, CDCl₃) 7.12-7.42 (4H, m, Ph), 5.80 (1H, m, CH═), 5.63 (1H, m, CH═), 5.38 (1H, m, CH—O), 3.99 (1H, d, J 7 Hz, CHaHb), 3.48 (1H, d, J 7 Hz, CHaHb), 3.00 (2H, m, CH₂), 2.08 (3H, s, CH₃), 2.00-2.22 (4H, m, 2×CH₂); m/z 276.4 [(M+NH₄)⁺, 100%].

### EXAMPLE 8

### 11-Hydroxy-7,10,11,12-tetrahydro-5H-benzocyclodecen-6-one

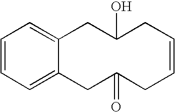

To a solution of acetic acid 11-oxo-5,6,7,10,11,12-hexahydro-benzocyclodecen-6-yl ester prepared in Example 7 (36 mg, 0.140 mmol) in dry methanol (5 ml) was added potassium carbonate (30 mg). The mixture was stirred at RT for 1 hour, quenched with water (10 ml) and extracted with DCM (2×15 ml). The organic layer was dried over Na₂SO₄, filtered, evaporated and purified by flash chromatography (silicagel, 30% ethyl acetate in hexane) to furnish the title compound in 83% yield (25 mg); 8H (300 MHz, CDCl₃) 7.14-7.34 (4H, m, Ph), 5.88 (1H, q, J 8 Hz, CH═), 5.62 (1H, q, J 8 Hz, CH═), 4.35 (1H, m, CH—O), 3.94 (1H, d, J 8 Hz, CHaHb), 3.54 (1H, d, J 8 Hz, CHaHb), 2.82-3.07 (4H, m, 2×CH2), 2.15 (2H, m, CH2), 1.87 (1H, bs, OH); m/z 275.3 [(M+CH₃COO)⁻, 100%], 234.3 [(M+NH₄)⁺, 100%].

### EXAMPLE 9

### 11-(Acetyloxy)-5,6,7,10,11,12-hexahydrobenzo[a][10]annulen-6-yl (2E)-3-(1-methyl-1H-imidazol-4-yl)prop-2-enoate

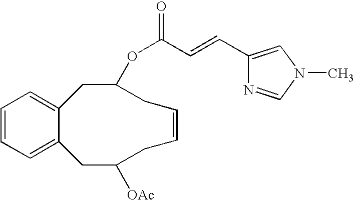

Acetic acid 11-hydroxy-5,6,7,10,11,12-hexahydro-benzocyclodec-8-en-6-yl ester prepared in Example 6 (22 mg, 0.085 mmol) and 3-(1′-Methyl-1′H-imidazol-4′-yl)-acrylic acid prepared as described in J. Am. Chem. Soc., Vol. 121, No. 28, p. 6563-6579, 1999 (65 mg) were stirred in DCM (10 ml) in the presence of DCC (106 mg) and 4-dimethylaminopyridine (106 mg) at RT under an atmosphere of nitrogen for 2 days. The mixture was quenched with a saturated solution of ammonium chloride (10 ml), dried over Na₂SO₄, filtered and evaporated. The residue was purified by HPLC to afford >98% pure title compound (0.80 mg); δ_{H} (300 MHz, CDCl₃) 7.60 (1H, d, J 15 Hz, CH═), 7.46 (1H, bs, CH═), 7.15-7.30 (4H, m, Ph), 7.09 (1H, bs, CH═), 6.60 (1H, d, J 15 Hz, CH═), 5.80 (2H, m, 2×CH), 5.42 (1H, m, CH—O), 5.35 (1H, m, CH—O), 3.23 (3H, s, CH₃), 3.12 (2H, td, J 1, 7 Hz, CH₂), 2.81 (2H, m, CH₂), 2.18 (3H, s, CH₃), 2.00-2.15 (4H, m, 2×CH₂); m/z 395.3 [(M+H)⁺, 100%].

Unequivocal assignment of cis stereochemistry of the double bond has been determined through X-Ray crystal structure.

### EXAMPLE 10

### 11-Hydroxy-5,6,7,10,11,12-hexahydrobenzo[a][10]annulen-6-yl (2E)-3-(1-methyl-1H-imidazol-4-yl)prop-2-enoate (Ia)

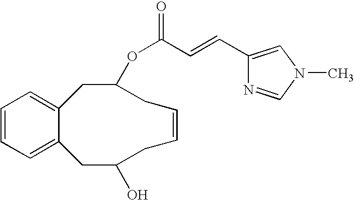

11-(Acetyloxy)-5,6,7,10,11,12-hexahydrobenzo[a][10]annulen-6-yl (2E)-3-(1-methyl-1H-imidazol-4-yl)prop-2-enoate prepared in Example 9, was treated with potassium carbonate as described in example 6, to give the title compound.

Operating as described in the previous examples, the following compounds are prepared:

### Ib) 11-(Acetyloxy)-5,6,7,10,11,12-hexahydrobenzo[a][10]annulen-6-yl (2E)-3-phenylprop-2-enoate

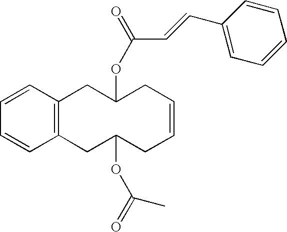

Molecular Weight=390.48

Exact Mass=390

Molecular Formula=C25H2604

Molecular Composition=C 76.90% H 6.71% O 16.39%

### Ic) 11-(Acetyloxy)-5,6,7,10,11,12-hexahydrobenzo[a][10]annulen-6-yl (2E)-3-(2-methyl-1,3-thiazol-4-yl)prop-2-enoate

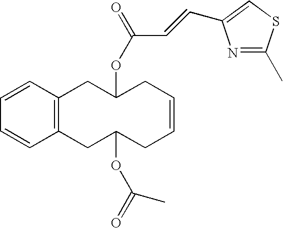

Molecular Weight=411.52

Exact Mass=411

Molecular Formula=C23H25NO4S

Molecular Composition=C 67.13% H 6.12% N 3.40% O 15.55% S 7.79%

### Id) 11-(Acetyloxy)-5,6,7,10,11,12-hexahydrobenzo[a][10]annulen-6-yl (2E)-3-(2-methyl-1,3-oxazol-4-yl)prop-2-enoate

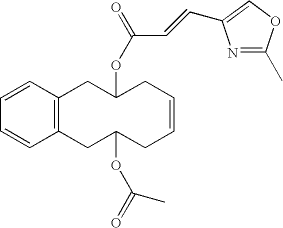

Molecular Weight=395.46

Exact Mass=395

Molecular Formula=C23H25NO5

Molecular Composition=C 69.86% H 6.37% N 3.54% O 20.23%

### Ie) 11-(Acetyloxy)-5,6,7,10,11,12-hexahydrobenzo[a][10]annulen-6-yl (2E)-3-pyridin-2-ylprop-2-enoate

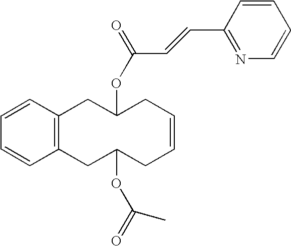

Molecular Weight=391.47

Exact Mass=391

Molecular Formula=C24H25NO4

Molecular Composition=C 73.64% H 6.44% N 3.58% O 16.35%

### If) 11-(Acetyloxy)-7-hydroxy-5,6,7,10,11,12-hexahydrobenzo[a][10]annulen-6-yl (2E)-3-(1-methyl-1H-imidazol-4-yl)prop-2-enoate

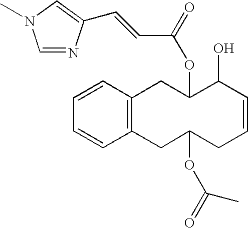

Molecular Weight=410.47

Exact Mass=410

Molecular Formula=C23H26N2O5

Molecular Composition=C 67.30% H 6.38% N 6.82% O 19.49%

### Ig) 11-(Acetyloxy)-7-oxo-5,6,7,10,11,12-hexahydrobenzo[a][10]annulen-6-yl (2E)-3-(1-methyl-1H-imidazol-4-yl)prop-2-enoate

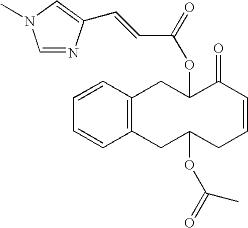

Molecular Weight=408.46

Exact Mass=408

Molecular Formula=C23H24N2O5

Molecular Composition=C 67.63% H 5.92% N 6.86% O 19.59%

### Ih) 7,11-Bis(acetyloxy)-5,6,7,10,11,12-hexahydrobenzo[a][10]annulen-6-yl (2E)-3-(1-methyl-1H-imidazol-4-yl)prop-2-enoate

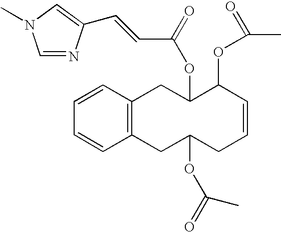

Molecular Weight=452.51

Exact Mass=452

Molecular Formula=C25H28N2O6

Molecular Composition=C 66.36% H 6.24% N 6.19% O 21.21%

### Ii) 11-(Acetyloxy)-5,6,7,8,9,10,11,12-octahydrobenzo[a][10]annulen-6-yl (2E)-3-(1-methyl-1H-imidazol-4-yl)prop-2-enoate

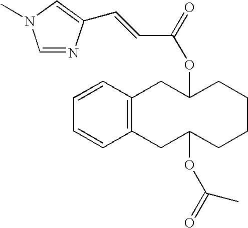

Molecular Weight=396.49

Exact Mass=396

Molecular Formula=C23H28N2O4

Molecular Composition=C 69.68% H 7.12% N 7.07% O 16.14%

### Il) 10-(Acetyloxy)-1a,2,3,4,9,10,11,11a-octahydro-1H-benzofalcyclopropa[f][10]annulen-3-yl (2E)-3-(1-methyl-1H-imidazol-4-yl)prop-2-enoate

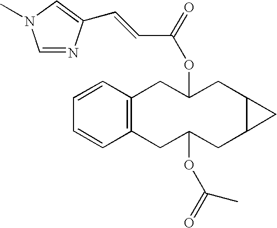

Molecular Weight=408.50

Exact Mass=408

Molecular Formula=C24H28N2O4

Molecular Composition=C 70.57% H 6.91% N 6.86% O 15.67%

### Im) 11-(acetyloxy)-10-hydroxy-5,6,7,10,11,12-hexahydrobenzo[a][10]annulen-6-yl (2E)-3-(1-methyl-1H-imidazol-4-yl)prop-2-enoate

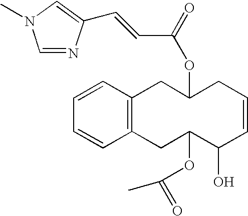

Molecular Weight=410.47

Exact Mass=410

Molecular Formula=C23H26N2O5

Molecular Composition=C 67.30% H 6.38% N 6.82% O 19.49%

### In) 10,11-Bis(acetyloxy)-5,6,7,10,11,12-hexahydrobenzo[a][10]annulen-6-yl (2E)-3-(1-methyl-1H-imidazol-4-yl)prop-2-enoate

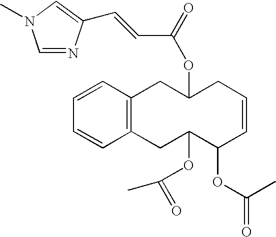

Molecular Weight=452.51

Exact Mass=452

Molecular Formula=C25H28N2O6

Molecular Composition=C 66.36% H 6.24% N 6.19% O 21.21%

### Io) 11-(Acetyloxy)-10-oxo-5,6,7,10,11,12-hexahydrobenzo[a][10]annulen-6-yl (2E)-3-(1-methyl-1H-imidazol-4-yl)prop-2-enoate

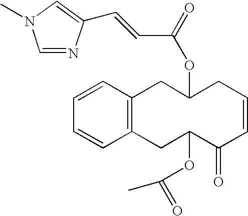

Molecular Weight=408.46

Exact Mass=408

Molecular Formula=C23H24N2O5

Molecular Composition=C 67.63% H 5.92% N 6.86% O 19.59%

### Ip) 11-Hydroxy-5,6,7,10,11,12-hexahydrobenzo[a][10]annulen-6-yl (2E)-3-(1-methyl-1H-imidazol-4-yl)prop-2-enoate

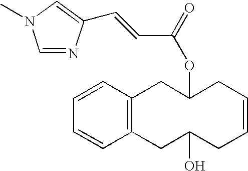

Molecular Weight=352.44

Exact Mass=352

Molecular Formula=C21H24N2O3

Molecular Composition=C 71.57% H 6.86% N 7.95% O 13.62%

### Iq) 11-Oxo-5,6,7,10,11,12-hexahydrobenzo[a][10]annulen-6-yl (2E)-3-(1-methyl-1H-imidazol-4-yl)prop-2-enoate

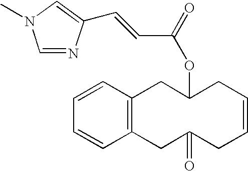

Molecular Weight=350.42

Exact Mass=350

Molecular Formula=C21H22N2O3

Molecular Composition=C 71.98% H 6.33% N 7.99% O 13.70%

### Ir) 11-Methylene-5,6,7,10,11,12-hexahydrobenzo[a][10]annulen-6-yl (2E)-3-(1-methyl-1H-imidazol-4-yl)prop-2-enoate

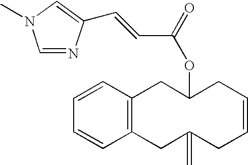

Molecular Weight=348.45

Exact Mass=348

Molecular Formula=C22H24N2O2

Molecular Composition=C 75.83% H 6.94% N 8.04% O 9.18%

### Is) 11-(2-Methoxy-2-oxoethylidene)-5,6,7,10,11,12-hexahydrobenzo[a][10]annulen-6-yl (2E)-3-(1-methyl-1H-imidazol-4-yl)prop-2-enoate

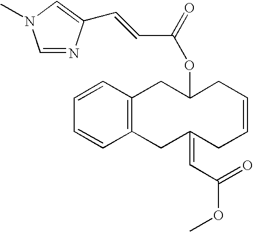

Molecular Weight=406.49

Exact Mass=406

Molecular Formula=C24H26N2O4

Molecular Composition=C 70.92% H 6.45% N 6.89% O 15.74%

### It) 11-(Methoxymethylene)-5,6,7,10,11,12-hexahydrobenzo[a][10]annulen-6-yl (2E)-3-(1-methyl-1H-imidazol-4-yl)prop-2-enoate

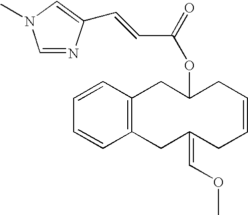

Molecular Weight=378.48

Exact Mass=378

Molecular Formula=C23H26N2O3

Molecular Composition=C 72.99% H 6.92% N 7.40% O 12.68%

### Iu) 11-(2-Ethoxy-2-oxoethylidene)-5,6,7,10,11,12-hexahydrobenzo[a][10]annulen-6-yl (2E)-3-(1-methyl-1 H-imidazol-4-yl)prop-2-enoate

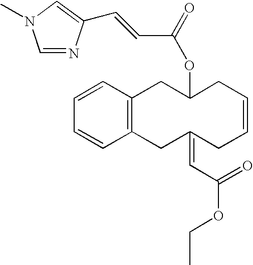

Molecular Weight=420.51

Exact Mass=420

Molecular Formula=C25H28N2O4

Molecular Composition=C 71.41% H 6.71% N 6.66% O 15.22%

### Iv) 11-Formyl-5,6,7,10,11,12-hexahydrobenzo[a][10]annulen-6-yl (2E)-3-(1-methyl-1H-imidazol-4-yl)prop-2-enoate

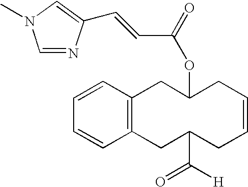

Molecular Weight=364.45

Exact Mass=364

Molecular Formula=C22H24N2O3

Molecular Composition=C 72.51% H 6.64% N 7.69% O 13.17%

### Iw) 11-(Hydroxymethyl)-5,6,7,10,11,12-hexahydrobenzo[a][10]annulen-6-yl (2E)-3-(1-methyl-1H-imidazol-4-yl)prop-2-enoate

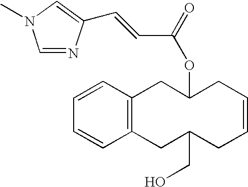

Molecular Weight=366.46

Exact Mass=366

Molecular Formula=C22H26N2O3

Molecular Composition=C 72.11% H 7.15% N 7.64% O 13.10%

### Iv) 11-{[(2-O-acetylpentolyranosyl)oxy]methyl}-5,6,7,10,11,12-hexahydrobenzo[a][10]annulen-6-yl (2E)-3-(1-methyl-1H-imidazol-4-yl)prop-2-enoate

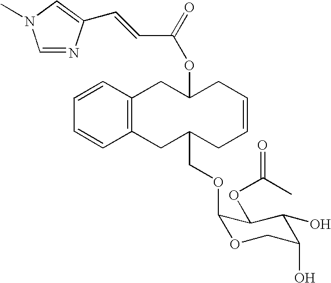

Molecular Weight=540.62

Exact Mass=540

Molecular Formula=C29H36N2O8

Molecular Composition=C 64.43% H 6.71% N 5.18% O 23.68%

### Iz) Ethyl 11-{[(2E)-3-(1-methyl-1H-imidazol-4-yl)prop-2-enoyl]oxy}-5,6,7,10,11,12-hexahydrobenzo[a][10]annulene-6-carboxylate

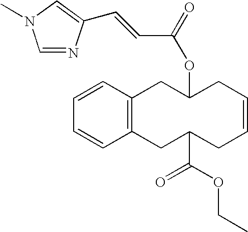

Molecular Weight=408.50

Exact Mass=408

Molecular Formula=C24H28N2O4

Molecular Composition=C 70.57% H 6.91% N 6.86% O 15.67%

### Iaa) Ethyl 7-hydroxy-11-{[(2E)-3-(1-methyl-1H-imidazol-4-yl)prop-2-enoyl]oxy}-7,10,11,12-tetrahydrobenzo[a][10]annulene-6-carboxylate

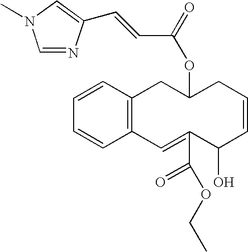

Molecular Weight=422.49

Exact Mass=422

Molecular Formula=C24H26N2O5

Molecular Composition=C 68.23% H 6.20% N 6.63% O 18.93%

### Ibb) 11-(Acetyloxy)-1-isopropyl-4-methyl-5,6,7,10,11,12-hexahydrobenzo[a][10]annulen-6-yl (2E)-3-(1-methyl-1H-imidazol-4-yl)prop-2-enoate

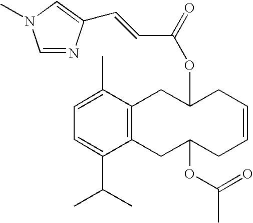

Molecular Weight=450.58

Exact Mass=450

Molecular Formula=C27H34N2O4

Molecular Composition=C 71.97% H 7.61% N 6.22% O 14.20%

### Icc) 7,11-Bis(acetyloxy)-1-isopropyl-4-methyl-5,6,7,10,11,12-hexahydrobenzo[a][10]annulen-6-yl (2E)-3-(1-methyl-1H-imidazol-4-yl)prop-2-enoate

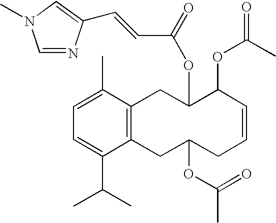

Molecular Weight=508.62

Exact Mass=508

Molecular Formula=C29H36N2O6

Molecular Composition=C 68.48% H 7.13% N 5.51% O 18.87%

### Idd) 10,11-Bis(acetyloxy)-1-isopropyl-4-methyl-5,6,7,10,11,12-hexahydrobenzo[a][10]annulen-6-yl (2E)-3-(1-methyl-1 H-imidazol-4-yl)prop-2-enoate

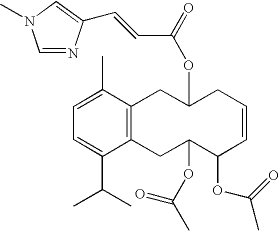

Molecular Weight=508.62

Exact Mass=508

Molecular Formula=C29H36N2O6

Molecular Composition=C 68.48% H 7.13% N 5.51% O 18.87%

### Iee) 11-(Acetyloxy)-1,4-dimethyl-5,6,7,10,11,12-hexahydrobenzo[a][10]annulen-6-yl (2E)-3-(1-methyl-1H-imidazol-4-yl)prop-2-enoate

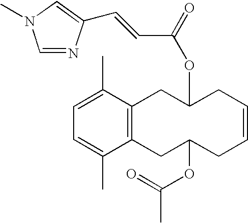

Molecular Weight=422.53

Exact Mass=422

Molecular Formula=C25H30N2O4

Molecular Composition=C 71.07% H 7.16% N 6.63% O 15.15%

### Iff) 7,11-bis(acetyloxy)-1,4-dimethyl-5,6,7,10,11,12-hexahydrobenzo[a][10]annulen-6-yl (2E)-3-(1-methyl-1H-imidazol-4-yl)prop-2-enoate

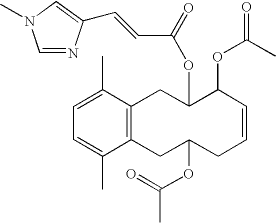

Molecular Weight=480.57

Exact Mass=480

Molecular Formula=C27H32N2O6

Molecular Composition=C 67.48% H 6.71% N 5.83% O 19.98%

### Igg) 10,11-Bis(acetyloxy)-1,4-dimethyl-5,6,7,10,11,12-hexahydrobenzo[a][10]annulen-6-yl (2E)-3-(1-methyl-1H-imidazol-4-yl)prop-2-enoate

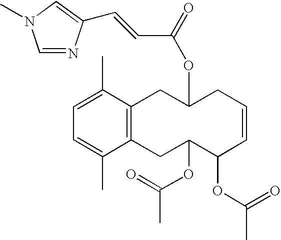

Molecular Weight=480.57

Exact Mass=480

Molecular Formula=C27H32N2O6

Molecular Composition=C 67.48% H 6.71% N 5.83% O 19.98%

1. A compound which is a benzocyclodecane of formula (I) 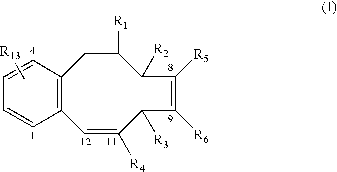
wherein: [symbol]
at positions 8-9 and 11-12 independently represents a single or double bond, —R₁ represents oxygen (═O), or a residue —OR₇, wherein R₇ represents hydrogen, linear or branched C₁-C₇ alkanoyl, benzoyl, C₁-C₁₀ alkyl, C₂-C₁₀ alkenyl or a residue of the formula 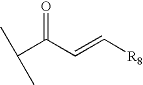
wherein R₈ is an optionally substituted aryl or heterocyclyl;
—R₂ and —R₃ independently represents hydrogen, oxygen atom (═O) or a residue —OR₉, wherein R₉ represents hydrogen, C₁-C₇ alkanoyl or benzoyl;
when [symbol] at position 11-12 represents a single bond, then —R₄ represents
oxygen atom (═O),
methylene (═CH₂),
═CHCOOR₁₀, wherein R₁₀ represents C₁-C₁₀ alkyl or optionally substituted aryl; ═CH(OCH₃),
or a residue of formula —OR₉, wherein R₉ is as defined above; —CH₂OR₁₁ wherein R₁₁ represents hydrogen or a sugar residue, —COR₁₂ wherein R₁₂ represents hydrogen, —OH or —OR₁₀, wherein R₁₀ is as defined above; or
when [symbol] at position 11-12 represents a double bond, then —R₄ represents a residue of formula —CH₂OR₁₁ or —COR₁₂ as defined above;
R₅ and —R₆ are both hydrogen atoms or, when [symbol] at position 8-9 represents a single bond, taken together with the carbon atoms to which they are attached form a cyclopropane ring;
R₁₃ represents hydrogen or from one to three substituents selected from C₁-C₆ alkyl, C₂-C₆ alkenyl, optionally substituted phenyl, phenyl C₁-C₆ alkyl, halogen, hydroxy, C₁-C₆ alkoxy, aryloxy, cyano, nitro, amino, C₁-C₁₀ alkylamino, arylamino, C₁-C₇ alkanoylamino, aroylamino, hydroxycarbonyl, aminocarbonyl, C₁-C₆ alkylcarbonyl, C₁-C₆ alkylaminosulfonyl and arylaminosulfonyl group;
with the provisos that if R₁ and R₄ are both oxygen atom (═O), then one of R₂, R₃, R₅, R₆ and R₁₃ is not hydrogen atom; or a pharmaceutically acceptable salt thereof.

2. A compound according to claim 1 wherein the benzocyclodecane has the formula IA 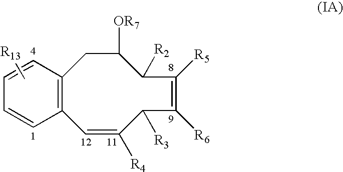
wherein: [symbol]
at positions 8-9 and 11-12 independently represents a single or double bond, R₇ represents a residue of the formula 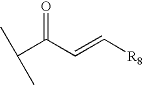
wherein R₈ is N-methyl imidazolyl, phenyl, methyl-thiazolyl, methyl-oxazolyl or pyridyl group;
one of —R₂ and —R₃ represents hydrogen and the other one is hydrogen or oxygen (═O), hydroxy or acetoxy group;
when [symbol] at position 11-12 represents a single bond, then —R₄ represents oxygen (═O), methylene (═CH₂), ═CHCOOR₁₀, wherein R₁₀ represents methyl or ethyl, ═CH(OCH₃), —CHO, hydroxy, acetoxy, or —CH₂OR₁₁ wherein R₁₁ represents hydrogen or a sugar residue having the formula 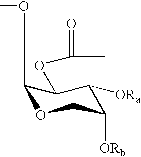
wherein Rₐ and R_{b} independently represent hydrogen, a hydroxy protecting group, or C₁-C₇ alkanoyl,
or
when [symbol] at position 11-12 represents a double bond, then —R₄ represents a residue of formula —O₂C₂H₅; and
R₅ and —R₆ are both hydrogen atoms or, when [symbol] at position 8-9 represents a single bond, taken together with the carbon atoms to which they are attached form a cyclopropane ring;
R₁₃ represents hydrogen atom, two methyl groups at positions 1 and 4, one methyl group at position 4 and one isopropyl group at position 1:

3. A compound as claimed in claim 2 wherein the substituent at ring position 6 is under the plane and R₈ is N-methyl imidazolyl group.

4. A process for preparing a compound of the formula I as defined in claim 1, which process comprises cyclizing a compound of formula II 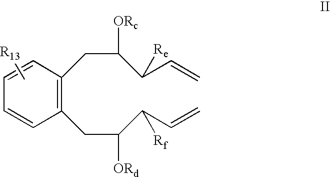
wherein R_{c} represents hydrogen, a hydroxy protecting group, C₁-C₇ alkanoyl or benzoyl or, taken together with Rₑ, forms an acetonide ring; R_{d} represents hydrogen, a hydroxy protecting group, C₁-C₆ alkanoyl, or benzoyl, or, taken together with R_{f}, forms an acetonide ring; R_{c} represents hydrogen atom and R_{f} represents hydrogen atom or a free or protected hydroxy group, or is linked to the adjacent OR_{d} substituent as defined above; R_{f} represents hydrogen atom and Rₑ represents hydrogen atom or a free or protected hydroxy group or is linked to the adjacent OR_{c} substituent as defined above; and, if desired, converting the resultant compound of formula I′: 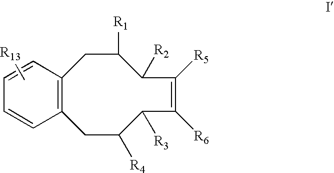
wherein R₁ is OR_{c}, R₂ is Rₑ, R₃ is R_{f}, R₄ is OR_{d}, in which R_{c}, R_{d}, Rₑ and R_{f} are as defined above and R₅ and R₆ are hydrogen atoms, into another different compound of formula I as defined in claim 1 by suitable reactions; and/or, if desired, recovering a single stereoisomer of a compound of formula I or I′ from a mixture of such stereoisomers; and/or if desired, converting a compound of formula I′ or I into a pharmaceutically acceptable salt therof; and/or, if desired converting a pharmaceutically acceptable salt of a compound of formula I or I′ into the corresponding free compound.

5. A process according to claim 4 characterized in that the cyclization is carried out through the Ring Closing Metathesis (RCM) reaction.

6. A process according to claim 5 in which RCM reaction is carried out in the presence of a Nolan and Grubb's catalyst.

7. A process for preparing a compound of the formula I′″: 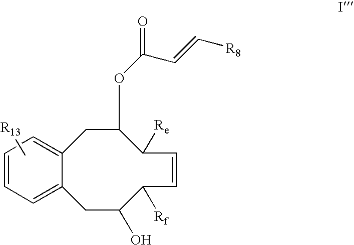
wherein Rₑ, R_{f} and R₁₃ are as defined in claim 4, and R₈ is as defined in claim 1, which process comprises deprotecting a compound of formula I′: 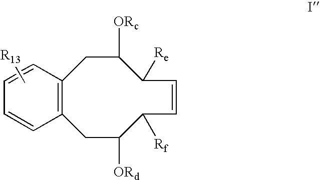
wherein R_{c}, Rₑ, R_{d}, R_{f} and R₁₃ are as defined in claim 4, condensing the resultant compound of formula I^{iv} 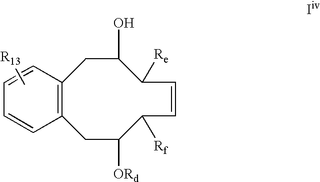
wherein Rₑ, R_{d}, R_{f} and R₁₃ are as defined above, with a compound of formula III or an activated form thereof: 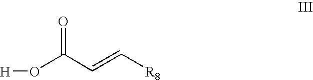
wherein R₈ is as above defined, optionally in presence of a condensing agent; and, if necessary, deprotecting the resultant compound of formula I^{v}. 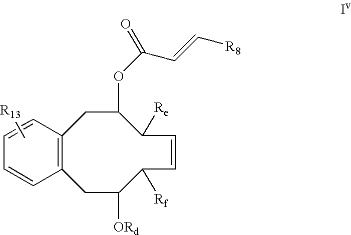
wherein R^{e}, R^{d}, R^{f}, R₈ and R₁₃ are as defined above, and R_{d} represents a hydroxy protecting group, C₁-C₆ alkanoyl, or benzoyl, or, taken together with R_{f}, forms an acetonide ring; to give the desired compound of formula I′″ as above defined.

8. A compound of formula II 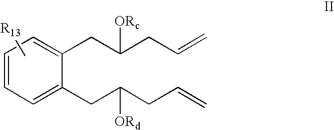
wherein R_{c} and R_{d} are hydrogen atoms or hydroxy protecting groups, and R₁₃ is as defined in claim 1.

9. A pharmaceutical composition which comprises a pharmaceutically acceptable diluent or carrier and, as an active ingredient, a compound as defined in claim 1.

10. A compound as defined in claim 1, 9-12 for use in a method of treatment of the human or animal body by therapy.

11. Use of a compound as defined in claim 1 in the manufacture of a medicament for use in the prevention, treatment and/or control of cancer.

12. A method of treating a patient in need of an antitumour agent, which method comprises the administration thereto of a compound as defined in claim 1.
